(19) United States
(12) Patent Application Publication
(10) Pub. No.: US 2025/0288687 A1
(43) Pub. Date: Sep. 18, 2025

(54) COMBINATION OF ANTIBODY-DRUG CONJUGATES AND DNMT INHIBITORS

(71) Applicants: DAIICHI SANKYO COMPANY, LIMITED, Tokyo (JP); AstraZeneca UK Limited, Cambridge (GB)

(72) Inventors: Daisuke OKAJIMA, Tokyo (JP); Reiko KAMEI, Tokyo (JP); Takami SUZUKI, Tokyo (JP)

(73) Assignees: DAIICHI SANKYO COMPANY, LIMITED, Tokyo (JP); AstraZeneca UK Limited, Cambridge (GB)

(21) Appl. No.: 19/222,453

(22) Filed: May 29, 2025

Related U.S. Application Data
(63) Continuation of application No. PCT/IB2023/062028, filed on Nov. 29, 2023.
(60) Provisional application No. 63/428,945, filed on Nov. 30, 2022.
(60) Provisional application No. 63/537,454, filed on Sep. 8, 2023.

(51) Int. Cl. A61K 47/68; A61K 31/7068; A61P 35/00; C07K 16/30

(52) U.S. Cl. CPC A61K 47/6851; A61K 31/7068; A61K 47/68037; A61P 35/00; C07K 16/30; C07K 2317/565

(57) ABSTRACT

A pharmaceutical product for administration of an antibody-drug conjugate in combination with a DNA methyltransferase (DNMT) inhibitor is provided. The antibody-drug conjugate is an antibody-drug conjugate in which a drug-linker represented by the following formula (wherein A represents the connecting position to an antibody) is conjugated to an antibody, in particular an anti-TROP2 antibody, via a thioether bond. Also provided is a therapeutic use and method wherein the antibody-drug conjugate and the DNMT inhibitor are administered in combination to a subject:

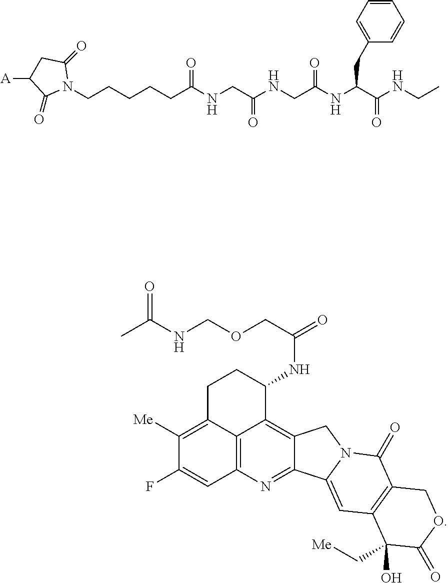

### CROSS-REFERENCE TO RELATED APPLICATIONS

This application is a Bypass Continuation of International Patent Application No. PCT/IB2023/062028, filed Nov. 29, 2023, which claims the benefit of priority to U.S. Provisional Application Nos. 63/428,945, filed Nov. 30, 2022, and 63/537,454, filed Sep. 8, 2023. The contents of these applications are hereby incorporated by reference in their entireties.

### SEQUENCE LISTING

The instant application contains a Sequence Listing which has been submitted via USPTO Patent Center is hereby incorporated by reference in its entirety. Said electronic copy, is named 122622-0239 SL.xml and is 23 kb in size.

### TECHNICAL FIELD

The present disclosure relates to a pharmaceutical product for administration of a specific antibody-drug conjugate, having an antitumor drug conjugated to an antibody, in particular an anti-TROP2 antibody, via a linker structure, in combination with a DNMT inhibitor, and to a therapeutic use and method wherein the specific antibody-drug conjugate and the DNMT inhibitor are administered in combination to a subject.

### BACKGROUND

Increasingly recognized in the field of cancer immunotherapy, DNA methyltransferase (DNMT) inhibitors are agents that inhibit DNA methyltransferase enzymes, inducing hypomethylation of DNA and growth inhibition or apoptosis in rapidly dividing cells. For example, decitabine (Dacogen®; 5-aza-2′-deoxycytidine) and azacitidine (Vidiza/Onureg®; 5-aza-cytidine) are nucleoside analogues that are used in the treatment acute myeloid leukemia (AML), myelodysplastic syndrome (MDS) and chronic myelomonocytic leukaemia (CMML).

Antibody-drug conjugates (ADCs), which are composed of a cytotoxic drug conjugated to an antibody, can deliver the drug selectively to and within cancer cells, leading to cancer cell death (Ducry, L., et al., Bioconjugate Chem. (2010) 21, 5-13; Alley, S. C., et al., Current Opinion in Chemical Biology (2010) 14, 529-537; Damle N. K. Expert Opin. Biol. Ther. (2004) 4, 1445-1452; Senter P. D., et al., Nature Biotechnology (2012) 30, 631-637; Burris HA., et al., J. Clin. Oncol. (2011) 29 (4): 398-405).

One such antibody-drug conjugate is datopotamab deruxtecan (Dato-DXd, DS-1062a), which is composed of a TROP2-targeting antibody and a derivative of exatecan. In particular, WO2015/098099 and WO2020/240467 provide detailed descriptions of exemplary TROP2-targeting antibody-drug conjugates, including datopotamab deruxtecan. Datopotamab deruxtecan has shown clinical efficacy in multiple tumor types, including lung cancer and breast cancer.

Inactivation of Schlafen 11 (SLFN11) in cancer cells has been shown to result in resistance to anticancer agents that cause DNA damage and replication stress. Thus, SLFN11 may serve as a determinant of sensitivity to different classes of DNA-damaging agents including but not restricted to topoisomerase I inhibitors (Zoppoli et al., PNAS 2012; 109:15030-35; Murai et al., Oncotarget 2016; 7:76534-50; Murai et al., Mol. Cell 2018; 69:371-84).

Zhao M, et al., AACR Cancer Res 2022; 82 (12_Suppl): Abstract nr 1791 reports that decitabine (a DNMT inhibitor) upregulates TROP2 and SLFN11 expression and enhances antitumor efficacy of sacituzumab govitecan (Trodelvy®, a humanized anti-TROP2 antibody conjugated with SN-38) in xenograft cell lines of metaplastic cancer origin and mesenchymal subtype breast cancer cell lines.

A need remains for improved therapeutic compositions and methods that can enhance efficacy of the existing cancer treating agents, increase durability of therapeutic response, improve tolerance to patients, reduce dose-dependent toxicity, and/or provide an alternative treatment of cancers exhibiting resistance or refractoriness to a previous cancer treatment. More specifically, there remains a need to identify further partnering combinations with antibody-drug conjugates, particularly with anti-TROP2 antibody-drug conjugates such as DS-1062a, to enhance their therapeutic potential. Therefore, it is desired to provide a medicine and treatment which can obtain a superior antitumor effect in the treatment of cancers, such as enhanced efficacy, increased durability of therapeutic response and/or reduced dose-dependent toxicity.

### SUMMARY OF DISCLOSURE

The present disclosure provides a pharmaceutical product which exhibits an excellent antitumor effect in the treatment of cancers, through administration of an antibody-drug conjugate, in particular an anti-TROP2 antibody-drug conjugate, in combination with a DNMT inhibitor. The present disclosure also provides a therapeutic use and method wherein the antibody-drug conjugate and the DNMT inhibitor are administered in combination to a subject.

Specifically, the present disclosure relates to the following [1] to:
[1] a pharmaceutical product comprising an antibody-drug conjugate and a DNMT inhibitor for administration in combination, wherein the antibody-drug conjugate is an antibody-drug conjugate in which a drug-linker represented by the following formula:

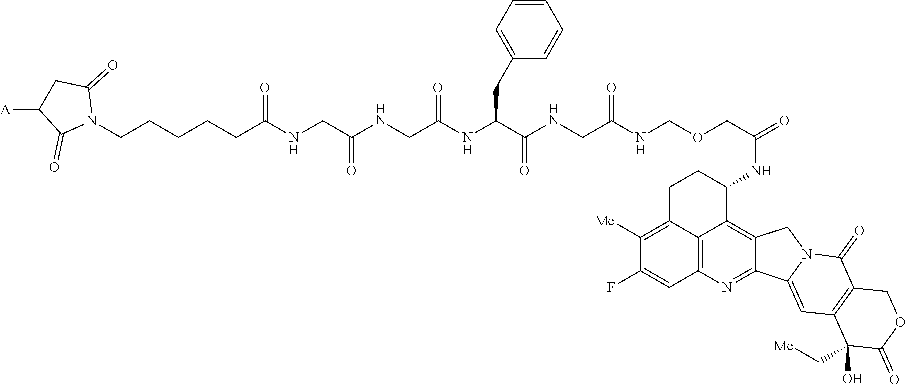

wherein A represents the connecting position to an antibody, is conjugated to an anti-TROP2 antibody via a thioether bond;
[2] the pharmaceutical product according to [1], wherein the anti-TROP2 antibody is an antibody comprising a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence represented by SEQ ID NO: 7 and CDRL3 consisting of an amino acid sequence represented by SEQ ID NO: 8;
[3] the pharmaceutical product according to [2], wherein the anti-TROP2 antibody is an antibody comprising a heavy chain comprising a heavy chain variable region consisting of an amino acid sequence represented by SEQ ID NO: 9 and a light chain comprising a light chain variable region consisting of an amino acid sequence represented by SEQ ID NO: 10;
[4] the pharmaceutical product according to [2] or [3], wherein the anti-TROP2 antibody is an antibody comprising a heavy chain consisting of an amino acid sequence represented by SEQ ID NO: 12 and a light chain consisting of an amino acid sequence represented by SEQ ID NO: 13;
[5] the pharmaceutical product according to [4], wherein the anti-TROP2 antibody lacks a lysine residue at the carboxyl terminus of the heavy chain;
[6] the pharmaceutical product according to any one of [1] to [5], wherein the average number of units of the drug-linker conjugated per antibody molecule in the antibody-drug conjugate is in the range of from 3.5 to 4.5;
[7] the pharmaceutical product according to any one of [1] to [5], wherein the antibody-drug conjugate is datopotamab deruxtecan (DS-1062a);
[8] the pharmaceutical product according to any one of [1] to [7], wherein the DNMT inhibitor is decitabine or azacitidine, or a pharmaceutically acceptable salt thereof;
[9] the pharmaceutical product according to [8], wherein the DNMT inhibitor is decitabine or a pharmaceutically acceptable salt thereof;
[10] the pharmaceutical product according to any one of [1] to [9] wherein the product is a composition comprising the antibody-drug conjugate and the DNMT inhibitor, for simultaneous administration;
[11] the pharmaceutical product according to any one of [1] to [9] wherein the product is a combined preparation comprising the antibody-drug conjugate and the DNMT inhibitor, for sequential or separate simultaneous administration;
[12] the pharmaceutical product according to any one of [1] to [11] wherein the DNMT inhibitor is administered in combination with a cytidine deaminase inhibitor;
[13] the pharmaceutical product according to [12], wherein the cytidine deaminase inhibitor is cedazuridine or a pharmaceutically acceptable salt thereof;
[14] the pharmaceutical product according to any one of [1] to [13], wherein the product is for treating cancer;
[15] the pharmaceutical product according to [14], wherein the cancer is at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma, cervical cancer, uterine cancer, testicular cancer, and renal cell carcinoma;
[16] the pharmaceutical product according to [15], wherein the cancer is colorectal cancer;
[17] the pharmaceutical product according to [15], wherein the cancer is lung cancer;
[18] the pharmaceutical product according to [17], wherein the lung cancer is non-small cell lung cancer;
[19] the pharmaceutical product according to [15], wherein the cancer is breast cancer;
[20] the pharmaceutical product according to any one of [14] to [19], wherein cancer cells of the cancer are SLFN11-deficient;
[21] the pharmaceutical product according to [20], wherein SLFN11 expression is lower in the cancer cells of a patient relative to the patient's SLFN11-expressing non-cancer cells;
[22] an antibody-drug conjugate for use, in combination with a DNMT inhibitor, in the treatment of cancer, wherein the antibody-drug conjugate and the DNMT inhibitor are as defined in any one of [1] to [9];
[23] the antibody-drug conjugate for the use according to [22], wherein the cancer is as defined in any one of [15] to [21];
[24] the antibody-drug conjugate for the use according to [22] or [23], wherein the use comprises administration of the antibody-drug conjugate and the DNMT inhibitor sequentially;
[25] the antibody-drug conjugate for the use according to [22] or [23], wherein the use comprises administration of the antibody-drug conjugate and the DNMT inhibitor separately and simultaneously;
[26] a method of treating cancer comprising administering an antibody-drug conjugate and a DNMT inhibitor as defined in any one of [1] to [9] in combination to a subject in need thereof; and
[27] the method according to [26], wherein the cancer is as defined in any one of [15] to [21].

### Advantageous Effects of Disclosure

The present disclosure provides a pharmaceutical product comprising a specified antibody-drug conjugate, having an antitumor drug conjugated to an antibody (in particular an anti-TROP2 antibody) via a linker structure, and a DNMT inhibitor, for administration in combination, and a therapeutic use and method wherein the specified antibody-drug conjugate and the DNMT inhibitor are administered in combination to a subject. Thus, the present disclosure provide a medicine and treatment which can obtain a superior antitumor effect in the treatment of cancers.

### BRIEF DESCRIPTION OF DRAWINGS

### [Anti-TROP2 Antibody]:

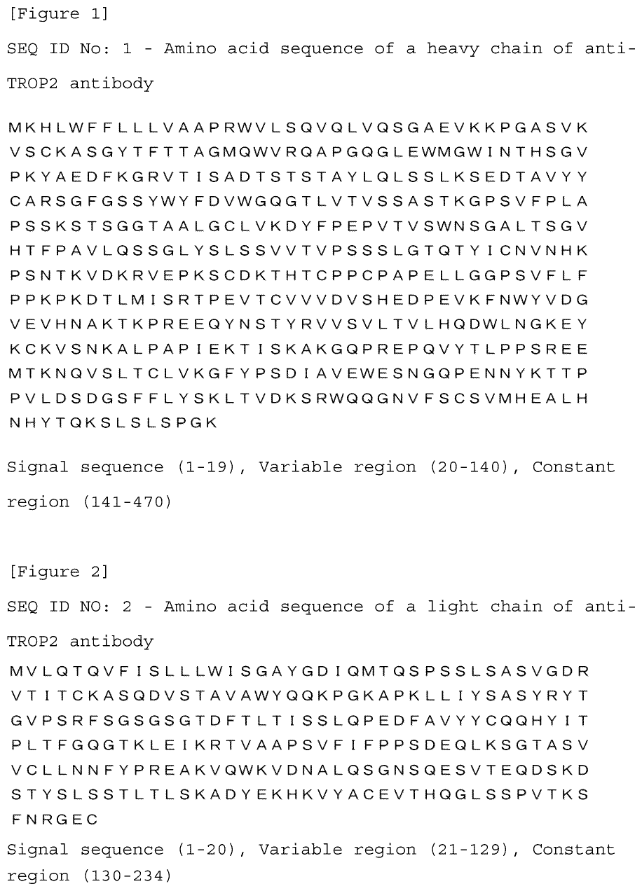

FIG. 1 is a diagram showing the amino acid sequence of a heavy chain of an anti-TROP2 antibody (SEQ ID NO: 1).

FIG. 2 is a diagram showing the amino acid sequence of a light chain of an anti-TROP2 antibody (SEQ ID NO: 2).

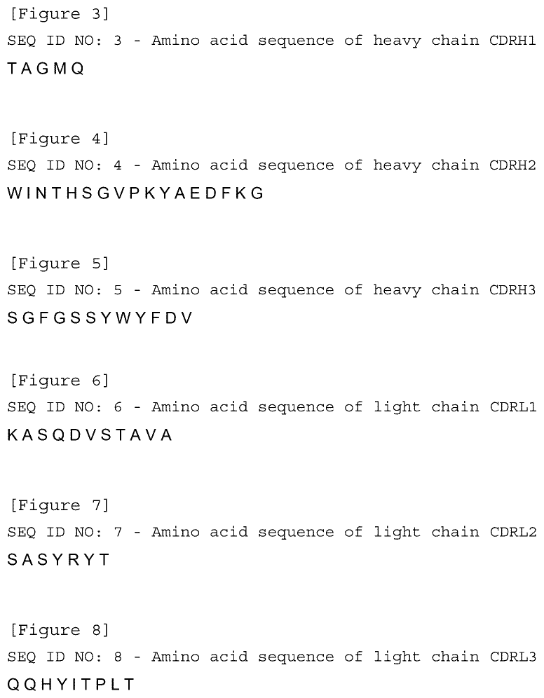

FIG. 3 is a diagram showing the amino acid sequence of a heavy chain CDRH1 (SEQ ID NO: 3 [=amino acid residues 50 to 54 of SEQ ID NO: 1]).

FIG. 4 is a diagram showing the amino acid sequence of a heavy chain CDRH2 (SEQ ID NO: 4 [=amino acid residues 69 to 85 of SEQ ID NO: 1]).

FIG. 5 is a diagram showing the amino acid sequence of a heavy chain CDRH3 (SEQ ID NO: 5 [=amino acid residues 118 to 129 of SEQ ID NO: 1]).

FIG. 6 is a diagram showing the amino acid sequence of a light chain CDRL1 (SEQ ID NO: 6 [=amino acid residues 44 to 54 of SEQ ID NO: 2]).

FIG. 7 is a diagram showing the amino acid sequence of a light chain CDRL2 (SEQ ID NO: 7 [=amino acid residues 70 to 76 of SEQ ID NO: 2]).

FIG. 8 is a diagram showing the amino acid sequence of a light chain CDRL3 (SEQ ID NO: 8 [=amino acid residues 109 to 117 of SEQ ID NO: 2]).

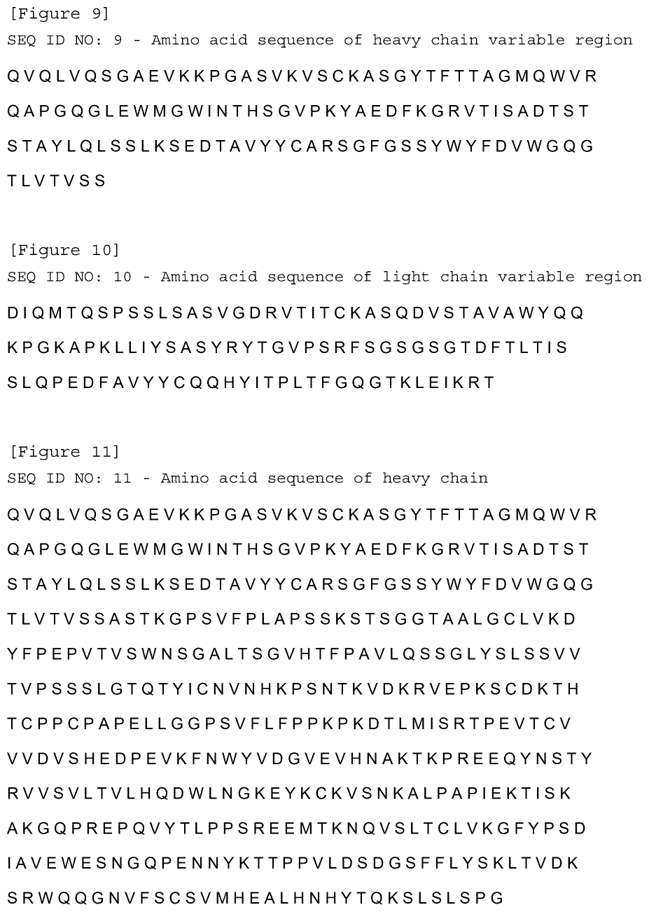

FIG. 9 is a diagram showing the amino acid sequence of a heavy chain variable region (SEQ ID NO: 9 [=amino acid residues 20 to 140 of SEQ ID NO: 1]).

FIG. 10 is a diagram showing the amino acid sequence of a light chain variable region (SEQ ID NO: 10 [=amino acid residues 21 to 129 of SEQ ID NO: 2]).

FIG. 11 is a diagram showing the amino acid sequence of a heavy chain (SEQ ID NO: 11 [=amino acid residues 20 to 469 of SEQ ID NO: 1]).

### [Experimental]:

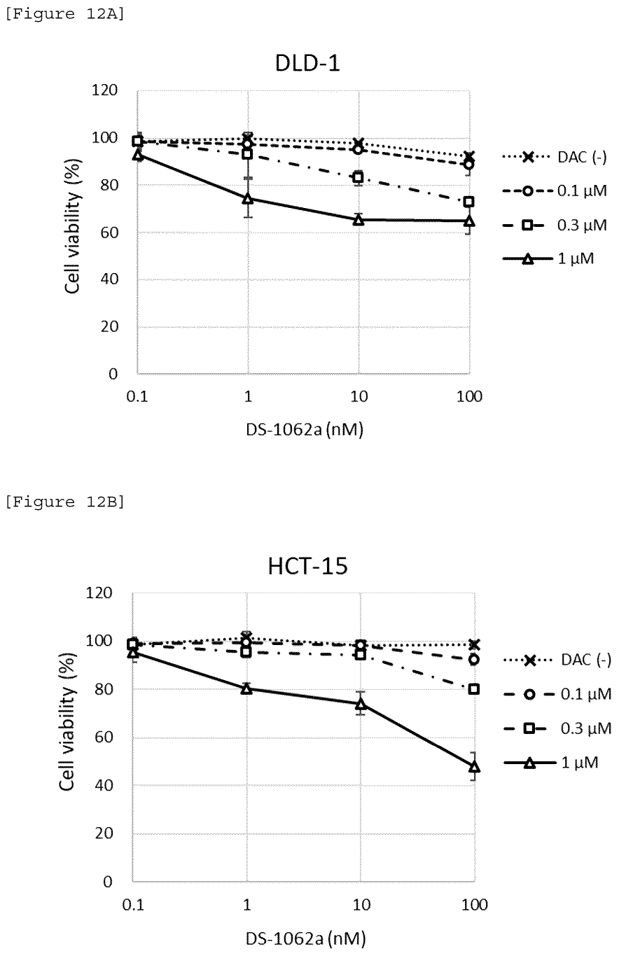

FIGS. 12A and 12B represent graphs showing cell growth inhibitory activity of DS-1062a in DLD-1 cells and HCT-15 cells respectively, with or without pre-treatment with decitabine (DAC).

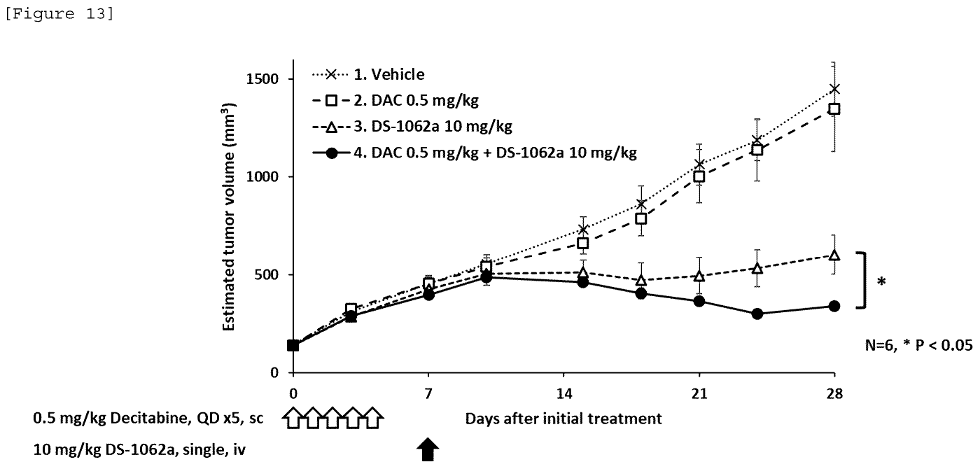

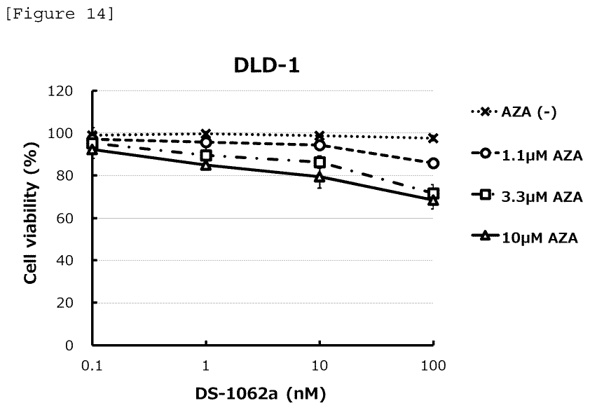

FIG. 13 represents a graph showing antitumor activity of DS-1062a in a DLD-1 xenograft mouse model with or without pre-treatment with decitabine (DAC).

FIG. 14 represents a graph showing cell growth inhibitory activity of DS-1062a in DLD-1 cells with or without pre-treatment with azacitidine (AZA).

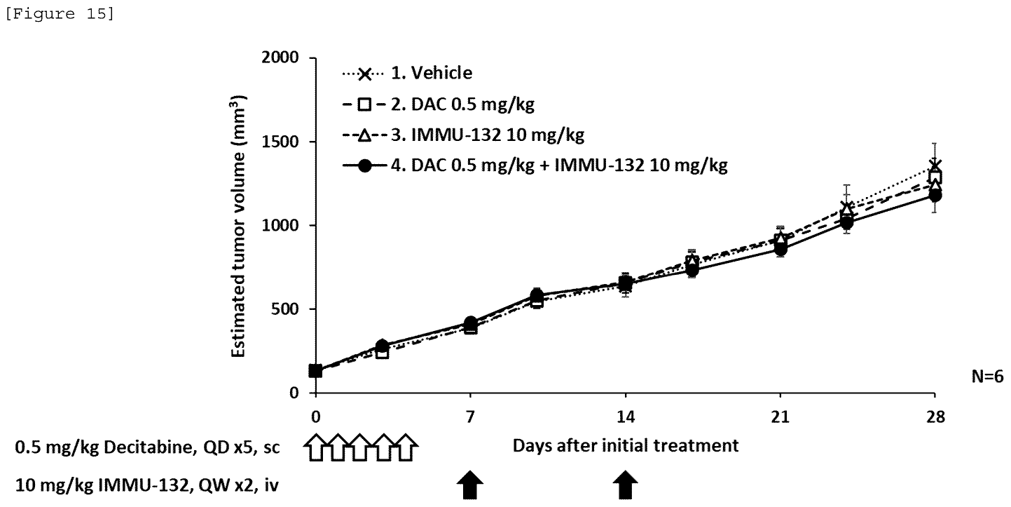

FIG. 15 represents a graph showing antitumor activity of IMMU-132 in a DLD-1 xenograft mouse model with or without pre-treatment with decitabine (DAC).

### DETAILED DESCRIPTION

In order that the present disclosure can be more readily understood, certain terms are first defined. Additional definitions are set forth throughout the detailed description.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Unless otherwise required by context, singular terms shall include pluralities and plural terms shall include the singular.

Units, prefixes, and symbols are denoted in their Système International de Unites (SI) accepted form. Numeric ranges are inclusive of the numbers defining the range.

It is understood that wherever aspects are described herein with the language “comprising”, otherwise analogous aspects described in terms of “consisting of” and/or “consisting essentially of” are also provided.

The terms “inhibit” and “inhibition” can refer to a decrease of about 10%, 20%, 30%, 40%, 50%, 60%, 70%, 80%, 90% or 100% in biological activity. Cellular proliferation can be assayed using art recognized techniques which measure rate of cell division, and/or the fraction of cells within a cell population undergoing cell division, and/or rate of cell loss from a cell population due to terminal differentiation or cell death (e.g., thymidine incorporation).

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical product” refers to a preparation which is in such form as to permit the biological activity of the active ingredients, either as a composition containing all the active ingredients (for simultaneous administration), or as a combination of separate compositions (a combined preparation) each containing at least one but not all of the active ingredients (for administration sequentially or simultaneously), and which contains no additional components which are unacceptably toxic to a subject to which the product would be administered. Such product can be sterile. By “simultaneous administration” is meant that the active ingredients are administered at the same time. By “sequential administration” is meant that the active ingredients are administered one after the other, in either order, at a time interval between the individual administrations. The time interval can be, for example, less than 24 hours, preferably less than 6 hours, more preferably less than 2 hours.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a subject is successfully “treated” for cancer according to the methods of the present disclosure if the patient shows, e.g., total, partial, or transient remission of a certain type of cancer.

The terms “cancer”, “tumor”, “cancerous”, and “malignant” refer to or describe the physiological condition in mammals that is typically characterized by unregulated cell growth. Examples of cancers include but are not limited to, breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, melanoma, cervical cancer, uterine cancer, testicular cancer, and renal cell carcinoma. Cancers include hematological malignancies such as acute myeloid leukemia, multiple myeloma, chronic lymphocytic leukemia, diffuse large B cell lymphoma, Burkitt's lymphoma, follicular lymphoma and solid tumors such as breast cancer, lung cancer, neuroblastoma and colon cancer.

The term “cytotoxic drug” as used herein is defined broadly and refers to a substance that inhibits or prevents the function of cells and/or causes destruction of cells (cell death), and/or exerts anti-neoplastic/anti-proliferative effects. For example, a cytotoxic drug prevents directly or indirectly the development, maturation, or spread of neoplastic tumor cells. The term includes also such agents that cause a cytostatic effect only and not a mere cytotoxic effect. The term includes chemotherapeutic agents as specified below.

The term “chemotherapeutic agent” is a subset of the term “cytotoxic drug” comprising natural or synthetic chemical compounds.

In accordance with the methods or uses of the present disclosure, compounds of the present disclosure may be administered to a patient to promote a positive therapeutic response with respect to cancer. The term “positive therapeutic response” with respect to cancer treatment refers to an improvement in the symptoms associated with the disease. For example, an improvement in the disease can be characterized as a complete response. The term “complete response” refers to an absence of clinically detectable disease with normalization of any previous test results.

Alternatively, an improvement in the disease can be categorized as being a partial response. A “positive therapeutic response” encompasses a reduction or inhibition of the progression and/or duration of cancer, the reduction or amelioration of the severity of cancer, and/or the amelioration of one or more symptoms thereof resulting from the administration of compounds of the present disclosure. In specific aspects, such terms refer to one, two or three or more results following the administration of compounds of the instant disclosure:
(1) a stabilization, reduction or elimination of the cancer cell population;
(2) a stabilization or reduction in cancer growth;
(3) an impairment in the formation of cancer;
(4) eradication, removal, or control of primary, regional and/or metastatic cancer;
(5) a reduction in mortality;
(6) an increase in disease-free, relapse-free, progression-free, and/or overall survival, duration, or rate;
(7) an increase in the response rate, the durability of response, or number of patients who respond or are in remission;
(8) a decrease in hospitalization rate,
(9) a decrease in hospitalization lengths,
(10) the size of the cancer is maintained and does not increase or increases by less than 10%, preferably less than 5%, preferably less than 4%, preferably less than 2%, and
(11) an increase in the number of patients in remission.
(12) a decrease in the number of adjuvant therapies (e.g., chemotherapy or hormonal therapy) that would otherwise be required to treat the cancer.

Clinical response can be assessed using screening techniques such as PET, magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy can experience the beneficial effect of an improvement in the symptoms associated with the disease.

As used herein, the term “the expression level of SLFN11 is” some amount, e.g. 0%, means that the stated amount of cancer cells in the patient's cancer tissue express SLEN11. Similarly, as used herein, the term “the expression level of SLEN11 is <” some amount, e.g. 10%, means that less than the stated amount of cancer cells in the patient's cancer tissue express SLFN11. The expression level of SLFN11 may be, for example, <25%, <20%, <15%, <10%, <9%, <8%, <7%, <6%, <5%, <4%, <3%, <2%, <1% or 0%.

As used herein, the term “SLFN11-deficient” refers to an expression level of SLFN11 in the relevant patient, animal, tissue, cell, etc. that is inadequate to exhibit the normal phenotype associated with the gene, or for the protein to exhibit its physiological function. In the context of preclinical models, cells or animals in which the SLFN11 gene is knocked out (KO) are examples of “SLFN11-deficient”.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain”, as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. As used herein, the terms “variable region” or “variable domain” are used interchangeably and are common in the art. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus. Those of skill in the art, however, would appreciate that the locations of the domains in a naturally occurring antibody can be modified in certain antibody-like binding protein formats without a loss of antigen-binding capability. Classes of human light chains are termed kappa and lambda light chains.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antibody fragment” refers to a portion of an intact or full-length chain or an antibody, generally the target binding or variable region. Examples of antibody fragments include, but are not limited to, F_{ab}, F_{ab′}, F_{(ab′)2} and Fᵥ fragments. As used herein, the term “functional fragment” is generally synonymous with “antibody fragment”, and with respect to antibodies, can refer to antibody fragments such as Fᵥ, F_{ab}, F_{(ab′)2}.

Reference to the numbering of amino acid residues described herein is performed according to the EU numbering system (also described in Kabat et al, Sequences of Proteins of Immunological Interest, 5th Ed. Public Health Service, National Institutes of Health, Bethesda, MD. (1991)).

A “monoclonal” antibody or antigen-binding fragment thereof refers to a homogeneous antibody or antigen-binding fragment population involved in the highly specific binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in manner including but not limited to by hybridoma, phage selection, recombinant expression, and transgenic animals.

The term “antigen” or “target antigen” as used herein refers to a molecule or a portion of a molecule that is capable of being recognized by and bound by binding proteins of the disclosure. The target antigen is capable of being used in an animal to produce antibodies capable of binding to an epitope of that antigen. A target antigen may have one or more epitopes.

The term “epitope” as used herein refers to a region or structural element of an antigen that is recognized and bound by a binding protein of the disclosure. More precisely, the epitope is the specific structure that is bound by the CDRs of the binding protein. Epitopes can comprise protein structural elements, carbohydrates or even portions of lipid structures found in membranes. A binding protein is said to specifically bind an antigen when it preferentially recognizes its antigen target in a complex mixture of proteins and/or macromolecules. The term “specifically binds” refers to a binding protein that specifically binds to a molecule or a fragment thereof (e.g., antigen). A binding protein that specifically binds a molecule or a fragment thereof may bind to other molecules with lower affinity as determined by, for example, immunoassays, BIAcore, or other assays known in the art. In particular, antibodies or fragments that specifically bind to at least one molecule or a fragment thereof can compete off molecules that bind non-specifically.

The term “antigen binding site” as used herein refers to a site created on the surface of a binding protein of the disclosure where an antigen or an epitope on an antigen is bound. The antigen binding site of the binding protein is typically described by reference to the loop structures created by complementarity determining regions (CDRs) of the binding protein.

### Description of Embodiments

Hereinafter, preferred modes for carrying out the present disclosure are described. The embodiments described below are given merely for illustrating one example of a typical embodiment of the present disclosure and are not intended to limit the scope of the present disclosure.

### 1. Antibody-Drug Conjugate

The antibody-drug conjugate used in the present disclosure is an antibody-drug conjugate in which a drug-linker represented by the following formula:

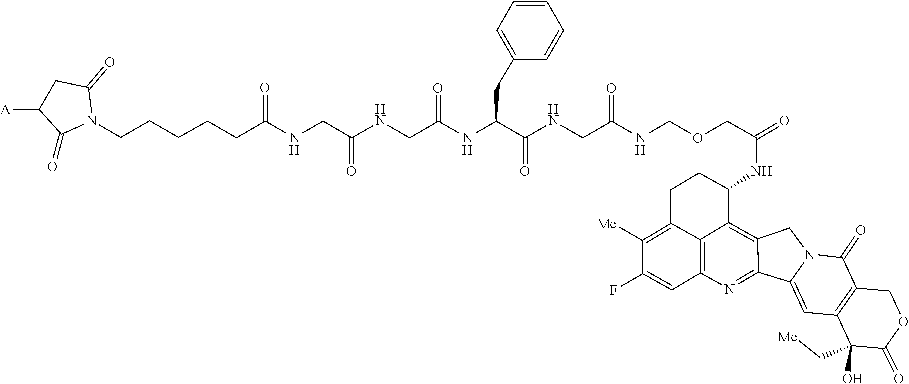

wherein A represents the connecting position to an antibody,
is conjugated to an antibody, in particular an anti-TROP2 antibody, via a thioether bond.

In the present disclosure, the partial structure consisting of a linker and a drug in the antibody-drug conjugate is referred to as a “drug-linker”. The drug-linker is connected to a thiol group (in other words, the sulfur atom of a cysteine residue) formed at an interchain disulfide bond site (two sites between heavy chains, and two sites between a heavy chain and a light chain) in the antibody.

The drug-linker of the present disclosure includes exatecan (IUPAC name: (1S,9S)-1-amino-9-ethyl-5-fluoro-1,2,3,9,12,15-hexahydro-9-hydroxy-4-methyl-10H, 13H-benzo[de] pyrano[3′,4′: 6,7] indolizino[1,2-b]quinolin-10,13-dione, (also expressed as chemical name: (1S,9S)-1-amino-9-ethyl-5-fluoro-2,3-dihydro-9-hydroxy-4-methyl-1H, 12H-benzo[de] pyrano[3′,4′: 6,7] indolizino[1,2-b] quinolin-10, 13 (9H, 15H)-dione)), which is a topoisomerase I inhibitor, as a component. Exatecan is a camptothecin derivative having an antitumor effect, represented by the following formula:

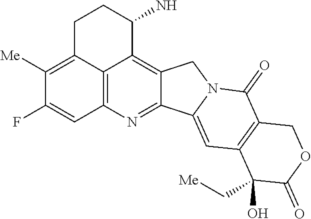 [SMILES: CC[C@@]1(O)C(=O)OCc2c1cc1n(c2=O)Cc2c-1nc1cc(F)c(C)c3c1c2[C@@H](N)CC3]

The antibody-drug conjugate used in the present disclosure can be also represented by the following formula:

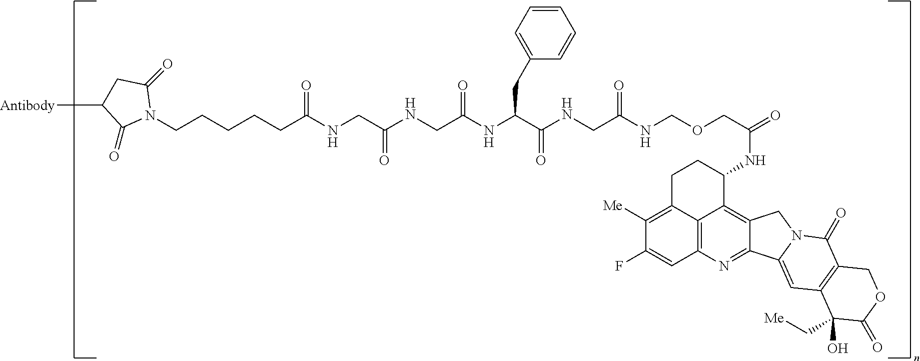

Here, the drug-linker is conjugated to an antibody (‘Antibody-’), in particular an anti-TROP2 antibody, via a thioether bond. The meaning of n is the same as that of what is called the average number of conjugated drug molecules (DAR; Drug-to-Antibody Ratio), and indicates the average number of units of the drug-linker conjugated per antibody molecule.

After migrating into cancer cells, the antibody-drug conjugate used in the present disclosure is cleaved at the linker portion to release a compound represented by the following formula:

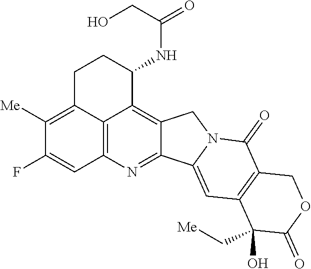 [SMILES: CC[C@@]1(O)C(=O)OCc2c1cc1n(c2=O)Cc2c-1nc1cc(F)c(C)c3c1c2[C@@H](NC(=O)CO)CC3]

### 2. Antibody in Antibody-Drug Conjugate

The antibody in the antibody-drug conjugate used in the present disclosure is an anti-TROP2 antibody, and may be derived from any species, preferably from a human, a rat, a mouse, or a rabbit. In cases when the antibody is derived from species other than human species, it is preferably chimerized or humanized using a well-known technique. The antibody may be a polyclonal antibody or a monoclonal antibody and is preferably a monoclonal antibody.

The antibody in the antibody-drug conjugate used in the present disclosure is an antibody preferably having a characteristic of being capable of targeting cancer cells, and is preferably an antibody possessing, for example, a property of recognizing a cancer cell, a property of binding to a cancer cell, a property of internalizing in a cancer cell, and/or cytocidal activity against cancer cells.

The binding activity of the antibody against cancer cells can be confirmed using flow cytometry. The internalization of the antibody into cancer cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282 December 2004), or (3) a Mab-ZAP assay using an immunotoxin binding to the therapeutic antibody wherein the toxin is released upon incorporation into cells to inhibit cell growth (Bio Techniques 28:162-165, January 2000). As the immunotoxin, a recombinant complex protein of a diphtheria toxin catalytic domain and protein G may be used.

The antitumor activity of the antibody can be confirmed in vitro by determining inhibitory activity against cell growth. For example, a cancer cell line overexpressing a target protein for the antibody is cultured, and the antibody is added at varying concentrations into the culture system to determine inhibitory activity against focus formation, colony formation, and spheroid growth. The antitumor activity can be confirmed in vivo, for example, by administering the antibody to a nude mouse with a transplanted cancer cell line highly expressing the target protein, and determining change in the cancer cell.

Since the compound conjugated in the antibody-drug conjugate exerts an antitumor effect, it is preferred but not essential that the antibody itself should have an antitumor effect. For the purpose of specifically and selectively exerting the cytotoxic activity of the antitumor compound against cancer cells, it is important and also preferred that the antibody should have the property of internalizing to migrate into cancer cells.

The anti-TROP2 antibody in the antibody-drug conjugate used in the present disclosure can be obtained by a procedure known in the art. For example, the antibody of the present disclosure can be obtained using a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen are fused with myeloma cells according to a method known in the art (e.g., Kohler and Milstein, Nature (1975) 256, p. 495-497; and Kennet, R. ed., Monoclonal Antibodies, p. 365-367, Plenum Press, N.Y. (1980)) to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The anti-TROP2 antibody in the antibody-drug conjugate used the present disclosure is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of a heterologous antibody into a human-derived antibody (Nature (1986) 321, pp. 522-525), and an antibody obtained by grafting a part of the amino acid residues of the framework of a heterologous antibody as well as the CDR sequence of the heterologous antibody to a human antibody by a CDR-grafting method (WO90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and light chain of a human antibody (see Tomizuka, K. et al., Nature Genetics (1997) 16, p. 133-143; Kuroiwa, Y. et. al., Nucl. Acids Res. (1998) 26, p. 3447-3448; Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p. 722-727, etc.) can be exemplified. As an alternative, an antibody obtained by phage display, the antibody being selected from a human antibody library (see Wormstone, I. M. et. al, Investigative Ophthalmology & Visual Science. (2002) 43 (7), p. 2301-2308; Carmen, S. et. al., Briefings in Functional Genomics and Proteomics (2002), 1 (2), p. 189-203; Siriwardena, D. et. al., Ophthalmology (2002) 109 (3), p. 427-431, etc.) can be exemplified.

In the antibody in the antibody-drug conjugate used in present disclosure, modified variants of the antibody are also included. The modified variant refers to a variant obtained by subjecting the antibody according to the present disclosure to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present disclosure, for example, an enzyme-labeled antibody, a fluorescence-labeled antibody, and an affinity-labeled antibody are also included in the meaning of the modified variant. Such a modified variant of the antibody according to the present disclosure is useful for improving the stability and blood retention of the antibody, reducing the antigenicity thereof, detecting or isolating an antibody or an antigen, and so on.

Further, by regulating the modification of a glycan which is linked to the antibody according to the present disclosure (glycosylation, defucosylation, etc.), it is possible to enhance antibody-dependent cellular cytotoxic activity. As the technique for regulating the modification of a glycan of antibodies, those disclosed in WO99/54342, WO00/61739, WO02/31140, WO2007/133855, WO2013/120066, etc. are known. However, the technique is not limited thereto. In the anti-TROP2 antibody according to the present disclosure, antibodies in which the modification of a glycan is regulated are also included.

It is known that a lysine residue at the carboxyl terminus of the heavy chain of an antibody produced in a cultured mammalian cell is deleted (Journal of Chromatography A, 705:129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360:75-83 (2007)). However, such deletion and modification of the heavy chain sequence do not affect the antigen-binding affinity and the effector function (the activation of complement, antibody-dependent cellular cytotoxicity, etc.) of the antibody. Therefore, in the anti-TROP2 antibody according to the present disclosure, antibodies subjected to such modification and functional fragments of the antibody are also included, and deletion variants in which one or two amino acids have been deleted at the carboxyl terminus of the heavy chain, variants obtained by amidation of deletion variants (for example, a heavy chain in which the carboxyl terminal proline residue has been amidated), and the like are also included. The type of deletion variant having a deletion at the carboxyl terminus of the heavy chain of the antibody according to the present disclosure is not limited to the above variants as long as the antigen-binding affinity and the effector function are conserved. The two heavy chains constituting the antibody according to the present disclosure may be of one type selected from the group consisting of a full-length heavy chain and the above-described deletion variant, or may be of two types in combination selected therefrom. The ratio of the amount of each deletion variant can be affected by the type of cultured mammalian cells which produce the antibody according to the present disclosure and the culture conditions; however, an antibody in which one amino acid residue at the carboxyl terminus has been deleted in both of the two heavy chains in the antibody according to the present disclosure can be exemplified as preferred.

As isotypes of the anti-TROP2 antibody according to the present disclosure, for example, IgG (IgG1, IgG2, IgG3, IgG4) can be exemplified, and IgG1 can be exemplified as preferred.

In the present disclosure, the term “anti-TROP2 antibody” refers to an antibody which binds specifically to TROP2 (TACSTD2: Tumor-associated calcium signal transducer 2; EGP-1), and preferably has an activity of internalization in TROP2-expressing cells by binding to TROP2.

Examples of the anti-TROP2 antibody include hTINA1-H1L1 (WO2015/098099), and datopotamab can be exemplified as preferred.

### 3. Production of Antibody-Drug Conjugate

A drug-linker intermediate for use in production of the antibody-drug conjugate according to the present disclosure is represented by the following formula:

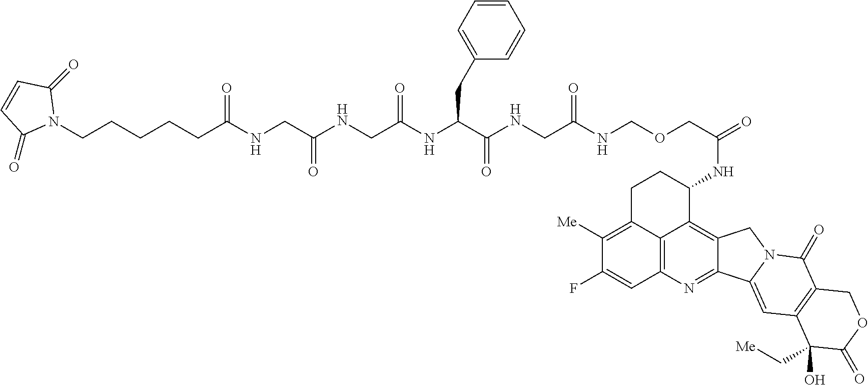 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

The drug-linker intermediate can be expressed as the chemical name N-[6-(2,5-dioxo-2,5-dihydro-1H-pyrrol-1-yl) hexanoyl]glycylglycyl-L-phenylalanyl-N-[(2-{[(1S,9S)-9-ethyl-5-fluoro-9-hydroxy-4-methyl-10,13-dioxo-2,3,9,10,13,15-hexahydro-1H, 12H-benzo[de] pyrano[3′,4′:6,7] indolizino[1,2-b]quinolin-1-yl] amino}-2-oxoethoxy)methyl]glycinamide, and can be produced with reference to descriptions in WO2014/057687, WO2015/098099, WO2019/044947 and so on.

The antibody-drug conjugate used in the present disclosure can be produced by reacting the above-described drug-linker intermediate and an anti-TROP2 antibody having a thiol group (also referred to as a sulfhydryl group).

An anti-TROP2 antibody having a sulfhydryl group can be obtained by a method well known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl) phosphine hydrochloride (TCEP) per interchain disulfide within the antibody and reacting with the antibody in a buffer solution containing a chelating agent such as ethylenediamine tetraacetic acid (EDTA), an anti-TROP2 antibody having a sulfhydryl group with partially or completely reduced interchain disulfides within the antibody can be obtained.

Further, by using 2 to 20 molar equivalents of the drug-linker intermediate per anti-TROP2 antibody having a sulfhydryl group, an antibody-drug conjugate in which 2 to 8 drug molecules are conjugated per antibody molecule can be produced.

The average number of conjugated drug molecules per anti-TROP2 antibody molecule of the antibody-drug conjugate produced can be determined, for example, by a method of calculation based on measurement of UV absorbance for the antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the anti-TROP2 antibody and the drug-linker intermediate and calculation of the average number of conjugated drug molecules per antibody molecule of the antibody-drug conjugate can be performed with reference to descriptions in WO2014/057687, WO2015/098099, WO2017/002776, WO2022/014698 and so on.

In the present disclosure, the term “anti-TROP2 antibody-drug conjugate” refers to an antibody-drug conjugate such that the antibody in the antibody-drug conjugate according to the disclosure is an anti-TROP2 antibody.

The anti-TROP2 antibody is preferably an antibody comprising a heavy chain comprising CDRH1 consisting of an amino acid sequence represented by SEQ ID NO: 3 [=an amino acid sequence consisting of amino acid residues 50 to 54 of SEQ ID NO: 1], CDRH2 consisting of an amino acid sequence represented by SEQ ID NO: 4 [=an amino acid sequence consisting of amino acid residues 69 to 85 of SEQ ID NO: 1], and CDRH3 consisting of an amino acid sequence represented by SEQ ID NO: 5 [=an amino acid sequence consisting of amino acid residues 118 to 129 of SEQ ID NO: 1], and a light chain comprising CDRL1 consisting of an amino acid sequence represented by SEQ ID NO: 6 [=an amino acid sequence consisting of amino acid residues 44 to 54 of SEQ ID NO: 2], CDRL2 consisting of an amino acid sequence represented by SEQ ID NO: 7 [=an amino acid sequence consisting of amino acid residues 70 to 76 of SEQ ID NO: 2], and CDRL3 consisting of an amino acid sequence represented by SEQ ID NO: 8 [=an amino acid sequence consisting of amino acid residues 109 to 117 of SEQ ID NO: 2],
more preferably an antibody comprising a heavy chain comprising a heavy chain variable region consisting of an amino acid sequence represented by SEQ ID NO: 9 [=an amino acid sequence consisting of amino acid residues 20 to 140 of SEQ ID NO: 1], and a light chain comprising a light chain variable region consisting of an amino acid sequence represented by SEQ ID NO: 10 [=an amino acid sequence consisting of amino acid residues 21 to 129 of SEQ ID NO: 2], and
even more preferably an antibody comprising a heavy chain consisting of an amino acid sequence represented by SEQ ID NO: 12 [=an amino acid sequence consisting of amino acid residues 20 to 470 of SEQ ID NO: 1] and a light chain consisting of an amino acid sequence represented by SEQ ID NO: 13 [=amino acid residues 21 to 234 of SEQ ID NO: 2], or an antibody comprising a heavy chain consisting of an amino acid sequence represented by SEQ ID NO: 11 [=an amino acid sequence consisting of amino acid residues 20 to 469 of SEQ ID NO: 1] and a light chain consisting of an amino acid sequence represented by SEQ ID NO: 13 [=amino acid residues 21 to 234 of SEQ ID NO: 2].

The average number of units of the drug-linker conjugated per antibody molecule in the anti-TROP2 antibody-drug conjugate is preferably 2 to 8, more preferably 3 to 5, even more preferably 3.5 to 4.5, and even more preferably about 4.

The anti-TROP2 antibody-drug conjugate can be produced with reference to descriptions in WO2015/098099, WO2017/002776 and WO2022/014698.

In preferred embodiments, the anti-TROP2 antibody-drug conjugate is datopotamab deruxtecan (DS-1062a).

### 4. DNA Methyltransferase (DNMT) Inhibitor

In the present disclosure, the terms “DNA methyltransferase inhibitor” and “DNMT inhibitor” refer to a compound that inhibits DNA methyltransferase enzymes. In some aspects, the DNMT inhibitor inhibits human DNA methyltransferase enzymes, including one or more of DNMT1, DNMT2, DNMT3a, and DNMT3b.

In some aspects, the DNA methyltransferase inhibitor is selected from decitabine (5-aza-2′-deoxycytidine), azacitidine (5-azacytidine), guadecitabine, 5,6-dihydro-5-azacytidine, fazarabine, 5-fluoro-2′-deoxycitidine, zebularine, hydralizine, procaine, procainamide, epigallocatechin gallate, psammaplin A, (S)-2-(1,3-dioxo-1,3-dihydro-isoindol-2-yl)-3-(1H-indol-3-yl)-propionic acid, and pharmaceutically acceptable salts thereof. Preferred DNA methyltransferase inhibitors include decitabine (5-aza-2′-deoxycytidine), azacitidine (5-azacytidine), 5,6-dihydro-5-azacytidine, fazarabine, 5-fluoro-2′-deoxycitidine, zebularine, and pharmaceutically acceptable salts thereof. Preferably the DNMT inhibitor is decitabine or azacitidine, or a pharmaceutically acceptable salt thereof, in particular decitabine or a pharmaceutically acceptable salt thereof.

### 5. Combination of Antibody-Drug Conjugate and DNMT Inhibitor

In a combination embodiment of the disclosure, the antibody-drug conjugate which is combined with the DNMT inhibitor is an antibody-drug conjugate in which the antibody is an anti-TROP2 antibody.

In an embodiment of the combination embodiment described above, the anti-TROP2 antibody comprises a heavy chain comprising CDRH1 consisting of an amino acid sequence represented by SEQ ID NO: 3 [=amino acid residues 50 to 54 of SEQ ID NO: 1], CDRH2 consisting of an amino acid sequence represented by SEQ ID NO: 4 [=amino acid residues 69 to 85 of SEQ ID NO: 1] and CDRH3 consisting of an amino acid sequence represented by SEQ ID NO: 5 [=amino acid residues 118 to 129 of SEQ ID NO: 1], and a light chain comprising CDRL1 consisting of an amino acid sequence represented by SEQ ID NO: 6 [=amino acid residues 44 to 54 of SEQ ID NO: 2], CDRL2 consisting of an amino acid sequence represented by SEQ ID NO: 7 [=amino acid residues 70 to 76 of SEQ ID NO: 2] and CDRL3 consisting of an amino acid sequence represented by SEQ ID NO: 8 [=amino acid residues 109 to 117 of SEQ ID NO: 2]. In another embodiment of the combination embodiment described above, the anti-TROP2 antibody comprises a heavy chain comprising a heavy chain variable region consisting of an amino acid sequence represented by SEQ ID NO: 9 [=amino acid residues 20 to 140 of SEQ ID NO: 1] and a light chain comprising a light chain variable region consisting of an amino acid sequence represented by SEQ ID NO: 10 [=amino acid residues 21 to 129 of SEQ ID NO: 2]. In another embodiment of the combination embodiment described above, the anti-TROP2 antibody comprises a heavy chain consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2]. In another embodiment of the combination embodiment described above, the anti-TROP2 antibody comprises a heavy chain consisting of an amino acid sequence represented by SEQ ID NO: 11 [=amino acid residues 20 to 469 of SEQ ID NO: 1] and a light chain consisting of an amino acid sequence represented by SEQ ID NO: 13 [=amino acid residues 21 to 234 of SEQ ID NO: 2]. In another embodiment of the combination embodiment described above, the anti-TROP2 antibody is datopotamab.

In a particularly preferred embodiment of the combination embodiment described above, the anti-TROP2 antibody-drug conjugate is datopotamab deruxtecan (DS-1062a) and the DNMT inhibitor is decitabine.

In another particularly preferred embodiment of the combination embodiment described above, the anti-TROP2 antibody-drug conjugate is datopotamab deruxtecan (DS-1062a) and the DNMT inhibitor is azacitidine.

In some aspects of the above combination embodiments, the antibody-drug conjugate and/or DNMT inhibitor are administered in further combination with one or more chemotherapeutic agents. In some aspects, the DNMT inhibitor is administered in further combination with a cytidine deaminase (CDA) inhibitor, preferably with cedazuridine or tetrahydrouridine, in particular with cedazuridine, preferably as an orally administered fixed-dose combination such as orally administered ASTX727 (Inqovi®: decitabine/cedazuridine).

### 6. Therapeutic Combined Use and Method

Described in the following are a pharmaceutical product and a therapeutic use and method wherein the anti-TROP2 antibody-drug conjugate according to the present disclosure and a DNMT inhibitor are administered in combination.

The pharmaceutical product and therapeutic use and method of the present disclosure may be characterized in that the antibody-drug conjugate and the DNMT inhibitor are separately contained as active components in different formulations, and are administered simultaneously or at different times, or characterized in that the antibody-drug conjugate and the DNMT inhibitor are contained as active components in a single formulation and administered.

In the pharmaceutical product and therapeutic method of the present disclosure, a single DNMT inhibitor used in the present disclosure can be administered in combination with the antibody-drug conjugate, or two or more different DNMT inhibitors can be administered in combination with the antibody-drug conjugate.

The pharmaceutical product and therapeutic method of the present disclosure can be used for treating cancer, and can be preferably used for treating at least one cancer selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, melanoma, cervical cancer, uterine cancer, testicular cancer, and renal cell carcinoma.

The presence or absence of tumor markers such as TROP2 tumor markers can be determined, for example, by collecting tumor tissue from a cancer patient to prepare a formalin-fixed, paraffin-embedded (FFPE) specimen and subjecting the specimen to a test for gene products (proteins), for example, with an immunohistochemical (IHC) method, a flow cytometer, or Western blotting, or to a test for gene transcription, for example, with an in situ hybridization (ISH) method, a quantitative PCR method (q-PCR), or microarray analysis, or by collecting cell-free circulating tumor DNA (ctDNA) from a cancer patient and subjecting the ctDNA to a test with a method such as next-generation sequencing (NGS).

The pharmaceutical product and therapeutic method of the present disclosure can be preferably used for a mammal, but are more preferably used for a human.

The antitumor effect of the pharmaceutical product and therapeutic method of the present disclosure can be confirmed by transplanting cancer cells to a test subject animal to prepare a model and measuring reduction in tumor volume or life-prolonging effect by application of the pharmaceutical product and therapeutic method of the present disclosure. And then, the effect of combined use of the antibody-drug conjugate used in the present disclosure and a DNMT inhibitor can be confirmed by comparing antitumor effect with single administration of the antibody-drug conjugate used in the present disclosure and that of the DNMT inhibitor.

The antitumor effect of the pharmaceutical product and therapeutic method of the present disclosure can be confirmed in a clinical trial using any of an evaluation method with Response Evaluation Criteria in Solid Tumors (RECIST), a WHO evaluation method, a Macdonald evaluation method, body weight measurement, and other approaches, and can be determined on the basis of indexes of complete response (CR), partial response (PR); progressive disease (PD), objective response rate (ORR), duration of response (DoR), progression-free survival (PFS), overall survival (OS), and so on.

By using the above methods, the superiority in antitumor effect of the pharmaceutical product and therapeutic method of the present disclosure to existing pharmaceutical products and therapeutic methods for cancer treatment can be confirmed.

The pharmaceutical product and therapeutic method of the present disclosure can delay development of cancer cells, inhibit growth thereof, and further kill cancer cells. These effects can allow cancer patients to be free from symptoms caused by cancer or achieve improvement in quality of life (QOL) of cancer patients and attain a therapeutic effect by sustaining the lives of the cancer patients. Even if the pharmaceutical product and therapeutic method of the present disclosure do not accomplish killing cancer cells, they can achieve higher QOL of cancer patients while achieving longer-term survival, by inhibiting or controlling the growth of cancer cells.

The pharmaceutical product of the present disclosure can be expected to exert a therapeutic effect by application as systemic therapy to patients, and additionally, by local application to cancer tissues.

The pharmaceutical product and therapeutic method of the present disclosure, in another aspect, provides for use as an adjuct in cancer therapy with ionizing radiation or other chemotherapeutic agents. For example, in the treatment of cancer, the treatment may comprise administering to a subject in need of treatment a therapeutically-effective amount of the pharmaceutical product, simultaneously or sequentially with ionizing radiation or other chemotherapeutic agents.

The pharmaceutical product and therapeutic method of the present disclosure can be used as adjuvant chemotherapy combined with surgery operation. The pharmaceutical product of the present disclosure may be administered for the purpose of reducing tumor size before surgical operation (referred to as preoperative adjuvant chemotherapy or neoadjuvant therapy), or may be administered for the purpose of preventing recurrence of tumor after surgical operation (referred to as postoperative adjuvant chemotherapy or adjuvant therapy).

In some embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535). BRCA1 and BRCA2 are known tumour suppressors whose wild-type alleles are frequently lost in tumours of heterozygous carriers (Jasin M., Oncogene, 21 (58), 8981-93 (2002); Tutt, et al., Trends Mol Med., 8 (12), 571-6, (2002)). The association of BRCA1 and/or BRCA2 mutations with breast cancer is well-characterised in the art (Radice, P. J., Exp Clin Cancer Res., 21 (3 Suppl), 9-12 (2002)). Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast, ovary, pancreas, prostate, hematological, gastrointestinal and lung cancer. In some embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Chappnis, P. O. and Foulkes, W. O., Cancer Treat Res, 107, 29-59 (2002); Janatova M., et al., Neoplasma, 50 (4), 246-505 (2003); Jancarkova, N., Ceska Gynekol., 68 {1), 11-6 (2003)). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

The pharmaceutical product of the present disclosure can be administered containing at least one pharmaceutically suitable ingredient. Pharmaceutically suitable ingredients can be suitably selected and applied from formulation additives or the like that are generally used in the art, in accordance with the dosage, administration concentration, or the like of the antibody-drug conjugate and the DNMT inhibitor used in the present disclosure. The antibody-drug conjugate used in the present disclosure can be administered, for example, as a pharmaceutical product containing a buffer such as histidine buffer, a vehicle such as sucrose and trehalose, and a surfactant such as Polysorbates 80 and 20. The antibody-drug conjugate used in the pharmaceutical product of the present disclosure can be preferably used as an injection, can be more preferably used as an aqueous injection or a lyophilized injection, and can be even more preferably used as a lyophilized injection. In the case that the pharmaceutical product containing the antibody-drug conjugate used in the present disclosure is an aqueous injection, the aqueous injection can be preferably diluted with a suitable diluent and then given as an intravenous infusion. Examples of the diluent can include dextrose solution and physiological saline, dextrose solution can be preferably exemplified, and 5% dextrose solution can be more preferably exemplified. In the case that the pharmaceutical product of the present disclosure is a lyophilized injection, a required amount of the lyophilized injection dissolved in advance in water for injection can be preferably diluted with a suitable diluent and then given as an intravenous infusion. Examples of the diluent can include dextrose solution and physiological saline, dextrose solution can be preferably exemplified, and 5% dextrose solution can be more preferably exemplified.

Examples of the administration route applicable to administration of the pharmaceutical product of the present disclosure can include intravenous, intradermal, subcutaneous, intramuscular, and intraperitoneal routes, and intravenous routes are preferred.

The size of the dose required for the therapeutic treatment of a particular disease state will necessarily be varied depending on the subject treated, the route of administration and the severity of the illness being treated. For further information on routes of administration and dosage regimes, reference may be made to Chapter 25.3 in Volume 5 of Comprehensive Medicinal Chemistry (Corwin Hansch; Chairman of Editorial Board), Pergamon Press 1990.

The anti-TROP2 antibody-drug conjugate used in the present disclosure can be administered to a human once at intervals of 1 to 180 days, and can be preferably administered once a week, once every 2 weeks, once every 3 weeks, or once every 4 weeks, and can be even more preferably administered once every 3 weeks. Also, the antibody-drug conjugate used in the present disclosure can be administered at a dose of about 0.001 to 100 mg/kg, and can be preferably administered at a dose of 0.8 to 12.4 mg/kg. For example, the anti-TROP2 antibody-drug conjugate can be administered once every 3 weeks at a dose of 0.27 mg/kg, 0.5 mg/kg, 1.0 mg/kg, 2.0 mg/kg, 4.0 mg/kg, 6.0 mg/kg, or 8.0 mg/kg, and can be preferably administered once every 3 weeks at a dose of 4.0 or 6.0 mg/kg.

The DNMT inhibitor may be administered in a suitable dose by any suitable route of administration.

In some aspects, the DNMT inhibitor is administered to the subject in a dose of about 0.1 to 10000 mg/m² of body surface area per administration, and can be preferably administered in a dose of 15 to 75 mg/m² per administration, and can be even more preferably administered in a dose of 15 or 20 mg/m².

In some aspects, a dose of the DNMT inhibitor is administered to the subject daily for 2 to 7 days at intervals of 3 to 6 weeks, and can be preferably administered daily for 3 or 5 days at intervals of 6 or 4 weeks, and can be even more preferably administered at 20 mg/m² daily for 5 days at intervals of 4 weeks.

In some aspects, the DNMT inhibitor is administered in combination with one or more other chemotherapeutic agents. In some aspects, the other chemotherapeutic agent is cedazuridine. In some aspects, the chemotherapeutic agent is pemetrexed.

In some aspects, the DNMT inhibitor disclosed herein may be formulated with a pharmaceutically acceptable carrier, excipient, or stabilizer, as pharmaceutical compositions. In certain aspects, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy”, 21st ed., Lippincott Williams & Wilkins, (2005), and in the “Physician's Desk Reference”, 60th ed., Medical Economics, Montvale, N.J. (2005). Pharmaceutically acceptable carriers can be selected that are suitable for the mode of administration, solubility, and/or stability desired or required.

In some aspects, therapeutic compositions can be formulated for particular routes of administration, such as oral, nasal, pulmonary, topical (including buccal and sublingual), rectal, vaginal, and/or parenteral administration. The terms “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Formulations of the disclosure that are suitable for topical or transdermal administration include powders, sprays, ointments, pastes, creams, lotions, gels, solutions, patches, and inhalants. The antibodies and other actives may be mixed under sterile conditions with a pharmaceutically acceptable carrier, and with any preservatives, buffers, or propellants which may be required (see, e.g., U.S. Pat. Nos. 7,378,110; 7,258,873; and 7,135,180; U.S. Patent Application Publication Nos. 2004/0042972 and 2004/0042971).

The formulations can be presented in unit dosage form and can be prepared by any method known in the art of pharmacy. Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. These dosages may be administered daily, weekly, biweekly, monthly, or less frequently, for example, biannually, depending on dosage, method of administration, disorder or symptoms to be treated, and individual subject characteristics. Dosages can also be administered via continuous infusion (such as through a pump). The administered dose may also depend on the route of administration. For example, subcutaneous administration may require a higher dosage than intravenous administration. As noted above, any commonly used dosing regimen (e.g., 1-10 mg/kg administered by injection or infusion daily or twice a week) may be adapted and suitable in the methods relating to treating human cancer patients.

### EXAMPLES

The present disclosure is specifically described in view of the examples shown below. However, the present disclosure is not limited to these. Further, it is by no means to be interpreted in a limited way.

### Example 1a: Production of Anti-TROP2 Antibody-Drug Conjugate (1)

In accordance with a production method described in WO2015/098099, WO2017/002776 and WO2022/014698 and using an anti-TROP2 antibody (an antibody comprising a heavy chain consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2]), an anti-TROP2 antibody-drug conjugate in which a drug-linker represented by the following formula:

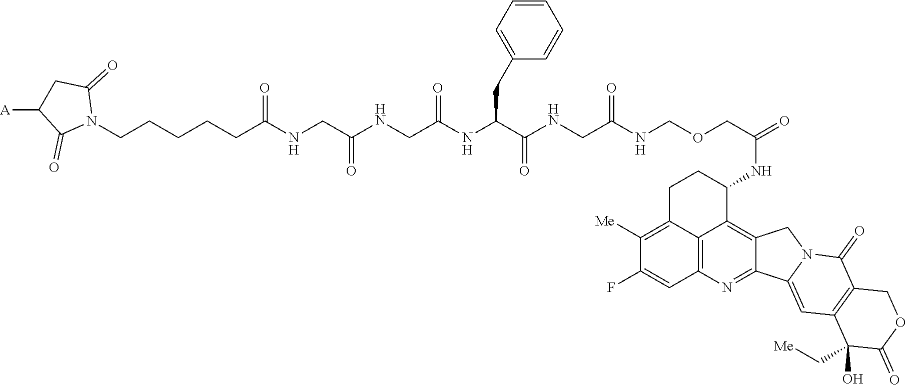

wherein A represents the connecting position to an antibody, is conjugated to the anti-TROP2 antibody via a thioether bond was produced (DS-1062a: datopotamab deruxtecan). The DAR of the antibody-drug conjugate (1) is 4.0.

### Example 1B: Production of Anti-TROP2 Antibody-Drug Conjugate (2)

Sacituzumab govitecan (IMMU-132) was prepared in accordance with a production method described in Example 12 of U.S. Pat. No. 7,999,083 and using hRS7 antibody (an antibody comprising a heavy chain consisting of an amino acid sequence represented by SEQ ID NO: 14 and a light chain comprising an amino acid sequence represented by SEQ ID NO: 15. The DAR of the antibody-drug conjugate (2) is 7.5.

### Example 2: Cell Growth Inhibition Study

Combination of Antibody-Drug Conjugate DS-1062a with Decitabine

Human colorectal cancer cell lines DLD-1 and HCT-15, obtained from American Type Culture Collection (ATCC), were incubated with RPMI1640 medium supplemented with 10% (v/v) heat-inactivated FBS, 1% (v/v) penicillin-streptomycin solution and 1 mM sodium pyruvate (Medium), with or without Decitabine (DAC) (Tokyo Chemical Industry Co., Ltd) at 1, 0.3 and 0.1 μM at 37° C. under 5% CO₂ for 3 days. The cells were harvested and re-seeded at 1000 cells/80 μL/well into 96-well black clear bottom plates, and incubated overnight at 37° C. under 5% CO₂. Then 20 μL of the DS-1062a solutions diluted with the Medium at the concentrations from 500 nM to 0.5 nM were added into the wells and incubated for 6 days for DLD-1 cells, and for 8 days for HCT-15 cells, respectively. The final concentrations of DS-1062a were 100, 10, 1 and 0.1 nM. After the incubation, the cellular ATP levels were measured using CellTiter-Glo Luminescent Cell Viability Assay (Promega) and a microplate reader.

Cell viability, defined as relative percent to the value of DS-1062a non-treated cells in each group treated with or without DAC, was assessed (N=1 (triplicate), mean±SD).

Individual cell viability (%) was calculated by the following equation:

$$Cell viability ( \% ) = 100 \times T / C$$

T: individual luminescence intensity of the wells treated with DS-1062a at each concentration
C: mean luminescence intensity of the wells treated without DS-1062a

### Results:

As shown in FIGS. 12A and 12B, the cell growth inhibitory activity of DS-1062a was enhanced in the DAC treated cells compared to the untreated cells, in the both DLD-1 and HCT-15 cell lines.

### Example 3: Antitumor Test

Combination of Antibody-Drug Conjugate DS-1062a with Decitabine

Female BALB/c-nu mice aged 5-6 weeks (The Jackson Laboratory Japan, Inc.) were used, following 4 days acclimatisation before entry into the study.

3×10⁶ of DLD-1 cells suspended in saline were implanted subcutaneously onto the flank of the mice. The major axis and the minor axis of the tumor were measured twice a week with an electronic digital caliper and the tumor volume was calculated by the following equation:

$$Tumor volume ( {mm}^{3} ) = 1 / 2 \times Major axis ( mm ) \times {[ Minor axis ( mm ) ]}^{2} .$$

When the tumor volume reached approximately 100-150 mm³, the tumor-bearing mice were randomly assigned to treatment groups (Day 0) as shown in Table 1:

TABLE 1

| Group | Treatment 1 | | Treatment 2 | |
|---|---|---|---|---|
| Vehicle | NT | NT | ABS | Single, IV |
| | | | | (Day 7) |
| Decitabine | Decitabine | QD × 5, SC | ABS | Single, IV |
| | 0.5 mg/kg | (Day 0-4) | | (Day 7) |
| DS-1062a | NT | NT | DS-1062a | Single, IV |
| | | | 10 mg/kg | (Day 7) |
| Decitabine + | Decitabine | QD × 5, SC | DS-1062a | Single, IV |
| DS-1062a | 0.5 mg/kg | (Day 0-4) | 10 mg/kg | (Day 7) |

QD: once per day (quaque die) dosing,
SC: subcutaneous dosing,
IV: intravenous dosing,
NT: non-treated
ABS: 10 mM Acetate buffer [pH 5.5], 5% sorbitol

Decitabine (Tokyo Chemical Industry Co., Ltd) was dissolved and diluted with Phosphate-buffered saline (PBS) and subcutaneously administered to the mice. DS-1062a solution was diluted with ABS buffer (10 mM Acetate buffer [pH5.5], 5% sorbitol) and intravenously administered to the tail vein of the mice. The dose of the compounds for each mouse was calculated based on the individual body weight on the day of dosing. Decitabine was subcutaneously administered at 0.5 mg/kg once a day from Day 0 to Day 4 (5 dosing in total). DS-1062a was intravenously administered at 10 mg/kg in a fluid volume of 10 mL/kg to the tail vein on Day 7 (Single dosing). Statistical significance between the ‘DS-1062a’ group and the ‘Decitabine+DS-1062a’ group was evaluated using Welch's t-test on the tumor volumes at the day of the final measurement (Day 28).

### Results:

As shown in FIG. 13, the antitumor activity of DS-1062a was significantly enhanced in the DAC pretreated group compared to the untreated group (P<0.05).

### Example 4: Cell Growth Inhibition Study

Combination of Antibody-Drug Conjugate DS-1062a with Azacitidine

Human colorectal cancer cell line DLD-1 was incubated with RPMI1640 medium supplemented with 10% (v/v) heat-inactivated FBS (Medium), with or without Azacitidine (AZA) at 10, 3.3 and 1.1 μM at 37° C. under 5% CO₂ for 3 days. The cells were harvested and re-seeded at 1000 cells/90 μL/well into a 96-well black clear bottom plate, and incubated overnight at 37° C. under 5% CO₂. Then 10 μL of the DS-1062a solutions diluted with the Medium at the concentrations from 1000 nM to 1 nM were added into the wells and incubated for 6 days. The final concentrations of DS-1062a were 100, 10, 1 and 0.1 nM. After the incubation, the cellular ATP levels were measured using CellTiter-Glo Luminescent Cell Viability Assay (Promega Corporation) and a microplate reader.

Cell viability, defined as relative percent to the value of DS-1062a non-treated cells in each group treated with or without AZA, was assessed (N=1 (triplicate), mean±SD).

Individual cell viability (%) was calculated by the following equation;

$$Cell viability ( \% ) = 100 \times T / C$$

T: individual luminescence intensity of the wells treated with DS-1062a at each concentration
C: mean luminescence intensity of the wells treated without DS-1062a

### Results:

As shown in FIG. 14, the cell growth inhibitory activity of DS-1062a was enhanced in the AZA treated cells compared to the AZA untreated cells.

### Example 5: Antitumor Test

Combination of Antibody-Drug Conjugate IMMU-132 with Decitabine

Female BALB/c-nu mice aged 5 weeks (The Jackson Laboratory Japan, Inc.) were used, following 5 days acclimatization before entry into the study.

3×10⁶ of DLD-1 cells suspended in saline were implanted subcutaneously onto the flank of the mice. The major axis and the minor axis of the tumor were measured twice a week with an electronic digital caliper and the tumor volume was calculated by the following equation:

$$Tumor volume ( {mm}^{3} ) = 1 / 2 \times Major axis ( mm ) \times {[ Minor axis ( mm ) ]}^{2} .$$

When the tumor volume reached approximately 100-150 mm³, the tumor-bearing mice were randomly assigned to the treatment groups (Day 0) as shown in Table 2.

Statistical significance between IMMU-132 group and Decitabine+IMMU-132 group was evaluated using Welch's t-test on the tumor volumes at the day of the final measurement (Day 28).

TABLE 2

| Group | Treatment 1 | | Treatment 2 | |
|---|---|---|---|---|
| Vehicle | NT | NT | ABS | QW × 2, IV |
| | | | | (Day 7, 14) |
| Decitabine | Decitabine | QD × 5, SC | ABS | QW × 2, IV |
| | 0.5 mg/kg | (Day 0-4) | | (Day 7, 14) |
| IMMU-132 | NT | NT | IMMU-132 | QW × 2, IV |
| | | | 10 mg/kg | (Day 7, 14) |
| Decitabine + | Decitabine | QD × 5, SC | IMMU-132 | QW × 2, IV |
| IMMU-132 | 0.5 mg/kg | (Day 0-4) | 10 mg/kg | (Day 7, 14) |

QD: once per day (quaque die) dosing,
QW: once per week (quaque week) dosing,
SC: subcutaneous dosing,
IV: intravenous dosing,
NT: non-treated
ABS: 10 mM Acetate buffer [pH 5.5], 5% sorbitol

Decitabine (DAC) was dissolved with Phosphate-buffered saline (PBS) and subcutaneously administered to the mice. IMMU-132 was dissolved with ABS buffer (10 mM Acetate buffer [pH5.5], 5% sorbitol) and intravenously administered to the tail vein of the mice. The dose volume of the compounds for each mouse was calculated based on the latest individual body weight measured within 2 days before dosing. DAC was subcutaneously administered at 0.5 mg/kg once a day from Day 0 to Day 4 (5 dosing in total). IMMU-132 was intravenously administered at 10 mg/kg to the tail vein on Day 7 and Day 14 (Weekly dosing×2).

### Results:

As shown in FIG. 15, the antitumor activity of IMMU-132 with DAC pre-treatment was not significantly different from that of IMMU-132 without DAC pre-treatment, while DS-1062a showed significantly enhanced antitumor activity with DAC pre-treatment as shown in FIG. 13 (Example 3).

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiments may be practiced in many ways and the claims include any equivalents thereof.

### FREE TEXT OF SEQUENCE LISTING

SEQ ID NO: 1—Amino acid sequence of a heavy chain of anti-TROP2 antibody

SEQ ID NO: 2—Amino acid sequence of a light chain of anti-TROP2 antibody

SEQ ID NO: 3—Amino acid sequence of a heavy chain CDRH1 [=amino acid residues 50 to 54 of SEQ ID NO: 1]

SEQ ID NO: 4—Amino acid sequence of a heavy chain CDRH2 [=amino acid residues 69 to 85 of SEQ ID NO: 1]

SEQ ID NO: 5—Amino acid sequence of a heavy chain CDRH3 [=amino acid residues 118 to 129 of SEQ ID NO: 1]

SEQ ID NO: 6—Amino acid sequence of a light chain CDRL1 [=amino acid residues 44 to 54 of SEQ ID NO: 2]

SEQ ID NO: 7—Amino acid sequence of a light chain CDRL2 [=amino acid residues 70 to 76 of SEQ ID NO: 2]

SEQ ID NO: 8—Amino acid sequence of a light chain CDRL3 [=amino acid residues 109 to 117 of SEQ ID NO: 2]

SEQ ID NO: 9—Amino acid sequence of a heavy chain variable region [=amino acid residues 20 to 140 of SEQ ID NO: 1]

SEQ ID NO: 10—Amino acid sequence of a light chain variable region [=amino acid residues 21 to 129 of SEQ ID NO: 2]

SEQ ID NO: 11—Amino acid sequence of a heavy chain [=amino acid residues 20 to 469 of SEQ ID NO: 1]

SEQ ID NO: 12—Amino acid sequence of a heavy chain [=amino acid residues 20 to 470 of SEQ ID NO: 1]

SEQ ID NO: 13—Amino acid sequence of a light chain [=amino acid residues 21 to 234 of SEQ ID NO: 2]

SEQ ID NO: 14—Amino acid sequence of a heavy chain of hRS7 antibody

SEQ ID NO: 15—Amino acid sequence of a light chain of hRS7 antibody

1. A pharmaceutical product comprising an antibody-drug conjugate and a DNMT inhibitor for administration in combination, wherein the antibody-drug conjugate is an antibody-drug conjugate in which a drug-linker represented by the following formula: 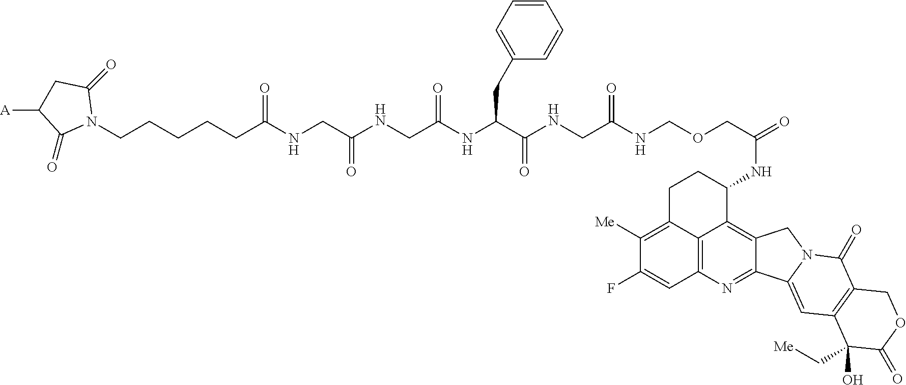
wherein A represents the connecting position to an antibody, is conjugated to an anti-TROP2 antibody via a thioether bond.

2. The pharmaceutical product according to claim 1, wherein the anti-TROP2 antibody is an antibody comprising a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence represented by SEQ ID NO: 7 and CDRL3 consisting of an amino acid sequence represented by SEQ ID NO: 8.

3. The pharmaceutical product according to claim 2, wherein the anti-TROP2 antibody is an antibody comprising a heavy chain comprising a heavy chain variable region consisting of an amino acid sequence represented by SEQ ID NO: 9 and a light chain comprising a light chain variable region consisting of an amino acid sequence represented by SEQ ID NO: 10.

4. The pharmaceutical product according to claim 2, wherein the anti-TROP2 antibody is an antibody comprising a heavy chain consisting of an amino acid sequence represented by SEQ ID NO: 12 and a light chain consisting of an amino acid sequence represented by SEQ ID NO: 13.

5. The pharmaceutical product according to claim 4, wherein the antibody lacks a lysine residue at the carboxyl terminus of the heavy chain.

6. The pharmaceutical product according to claim 1, wherein the average number of units of the drug-linker conjugated per antibody molecule in the antibody-drug conjugate is in the range of from 3.5 to 4.5.

7. The pharmaceutical product according to claim 1, wherein the antibody-drug conjugate is datopotamab deruxtecan (DS-1062a)

8. The pharmaceutical product according to claim 1, wherein the DNMT inhibitor is decitabine or azacitidine, or a pharmaceutically acceptable salt thereof.

9. The pharmaceutical product according to claim 8, wherein the DNMT inhibitor is decitabine or a pharmaceutically acceptable salt thereof.

10. The pharmaceutical product according to claim 1, wherein the product is a composition comprising the antibody-drug conjugate and the DNMT inhibitor, for simultaneous administration.

11. The pharmaceutical product according to claim 1, wherein the product is a combined preparation comprising the antibody-drug conjugate and the DNMT inhibitor, for sequential or separate simultaneous administration.

12. The pharmaceutical product according to claim 1, wherein the DNMT inhibitor is administered in combination with a cytidine deaminase inhibitor.

13. The pharmaceutical product according to claim 12, wherein the cytidine deaminase inhibitor is cedazuridine or a pharmaceutically acceptable salt thereof.

14. The pharmaceutical product according to claim 1, wherein the product is for treating cancer.

15. The pharmaceutical product according to claim 14, wherein the cancer is at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma, cervical cancer, uterine cancer, testicular cancer, and renal cell carcinoma.

16. The pharmaceutical product according to claim 15, wherein the cancer is colorectal cancer.

17. The pharmaceutical product according to claim 15, wherein the cancer is lung cancer.

18. The pharmaceutical product according to claim 17, wherein the lung cancer is non-small cell lung cancer.

19. The pharmaceutical product according to claim 15, wherein the cancer is breast cancer.

20. The pharmaceutical product according to claim 14, wherein cancer cells of the cancer are SLEN11-deficient.

21. The pharmaceutical product according to claim 20, wherein SLEN11 expression is lower in the cancer cells of a patient relative to the patient's SLFN11-expressing non-cancer cells.

22. An antibody-drug conjugate for use, in combination with a DNMT inhibitor, in the treatment of cancer, wherein the antibody-drug conjugate and the DNMT inhibitor are as defined in claim 1.

23. The antibody-drug conjugate for the use according to claim 21, wherein the cancer is at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma, cervical cancer, uterine cancer, testicular cancer, and renal cell carcinoma.

24. The antibody-drug conjugate for the use according to claim 22, wherein the use comprises administration of the antibody-drug conjugate and the DNMT inhibitor sequentially.

25. The antibody-drug conjugate for the use according to claim 22, wherein the use comprises administration of the antibody-drug conjugate and the DNMT inhibitor separately and simultaneously.

26. A method of treating cancer comprising administering an antibody-drug conjugate and a DNMT inhibitor as defined in claim 1 in combination to a subject in need thereof.

27. The method according to claim 26, wherein the cancer is at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma, cervical cancer, uterine cancer, testicular cancer, and renal cell carcinoma.


SEQUENCE LISTING (15 sequences)

SEQ ID NO: 1 - protein, 470 aa, synthetic construct
  REGION 1..470: note = Heavy chain of anti-TROP2 antibody
MKHLWFFLLLVAAPRWVLSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 234 aa, synthetic construct
  REGION 1..234: note = Light chain of anti-TROP2 antibody
MVLQTQVFISLLLWISGAYGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 5 aa, synthetic construct
  REGION 1..5: note = CDRH1 of heavy chain of anti-TROP2 antibody
TAGMQ

SEQ ID NO: 4 - protein, 17 aa, synthetic construct
  REGION 1..17: note = CDRH2 of heavy chain of anti-TROP2 antibody
WINTHSGVPKYAEDFKG

SEQ ID NO: 5 - protein, 12 aa, synthetic construct
  REGION 1..12: note = CDRH3 of heavy chain of anti-TROP2 antibody
SGFGSSYWYFDV

SEQ ID NO: 6 - protein, 11 aa, synthetic construct
  REGION 1..11: note = CDRL1 of light chain of anti-TROP2 antibody
KASQDVSTAVA

SEQ ID NO: 7 - protein, 7 aa, synthetic construct
  REGION 1..7: note = CDRL2 of light chain of anti-TROP2 antibody
SASYRYT

SEQ ID NO: 8 - protein, 9 aa, synthetic construct
  REGION 1..9: note = CDRL3 of light chain of anti-TROP2 antibody
QQHYITPLT

SEQ ID NO: 9 - protein, 121 aa, synthetic construct
  REGION 1..121: note = Variable region of heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 10 - protein, 109 aa, synthetic construct
  REGION 1..109: note = Variable region of light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRT

SEQ ID NO: 11 - protein, 450 aa, synthetic construct
  REGION 1..450: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 451 aa, synthetic construct
  REGION 1..451: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  REGION 1..214: note = Light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 451 aa, synthetic construct
  REGION 1..451: note = Heavy chain of hRS7 antibody
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, synthetic construct
  REGION 1..214: note = Light chain of hRS7 antibody
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC